(19) United States
(12) Patent Application Publication
(10) Pub. No.: US 2026/0035647 A1
(43) Pub. Date: Feb. 5, 2026

(54) DISCHARGE SYSTEM FOR CELL CULTURE WELLS AND METHOD FOR CULTURING CELLS

(71) Applicants: POLITECNICO DI MILANO, Milano (IT); FONDAZIONE CENTRO SAN RAFFAELE, Milano (IT)

(72) Inventors: Elena BIANCHI, Milano (IT); Oronza Antonietta BOTRUGNO, Milano (IT); Paola DESTEFANO, Milano (IT); Gabriele Angelo DUBINI, Milano (IT); Giovanni TONON, Milano (IT); Giulia VENTURINI, Milano (IT)

(21) Appl. No.: 18/997,704

(22) PCT Filed: May 4, 2023
(86) PCT No.: PCT/IB23/54652

§ 371 Date: Jan. 22, 2025

(30) Foreign Application Priority Data
Jul. 22, 2022 (IT) 102022000015453

(51) Int. Cl. C12M 1/00; C12M 1/32

(52) U.S. Cl. CPC C12M 29/00; C12M 23/12; C12M 29/10

(57) ABSTRACT

The present invention relates to a discharge system for cell culture wells, comprising an outlet (1) comprising a proximal portion (10) and a distal portion (11, 21), wherein said proximal portion (10) comprises a capillary which engages on said distal portion (11, 21), said capillary having an inlet and an outlet, wherein said outlet engages on said distal portion which is open and has an outlet opening with a greater hydraulic diameter than the hydraulic diameter of said capillary. The present invention further relates to a method for dynamically culturing cells and/or tissues using said discharge system.

### BACKGROUND ART

Large-scale biological assays are becoming more widespread. More and more often, tests are needed, which use cell models reproducing the microenvironment—in terms of architecture, biomechanics, and biochemistry—of the tissue from which said cells derive as closely as possible.

The final objectives are to evaluate how a tissue responds to various stimuli: physiological (for example, growth factors), pathological (microorganisms, inflammatory factors . . . ) or therapeutic, including new aspects of drug resistance; and to develop personalized therapeutic approaches.

Such tests require or are reinforced by the availability of methodologies for:
i) the manipulation of cells/particles, analytes and reagents in liquid and/or gel phases;
ii) the controlled seeding of fluorescent/bioluminescent molecules/cells/particles;
iii) the controlled immobilization of said cells/particles for the purpose of analysis;
iv) the maintenance of cell viability and cell function for periods of time sufficient for analysis;
v) the controlled exposure to one or more actives for the purpose of assessing the effect thereof on the sample.

Important advances have been made in dispensing volumes of liquids of the order of microliters with efficiency and reproducibility, which advances have allowed increasing the number of samples and minimizing the biological sample required, an especially important factor when taking material from the patient.

Several studies have shown that static culture is inadequate since it is not capable of in vitro replicating the conditions of nourishment and exposure to external agents or drugs occurring in vivo. Under physiological conditions, the blood flow allows the continuous supply of nutrients and the elimination of waste substances. Moreover, the concentration of added compounds does not remain constant over time, as it is regulated by metabolic and excretion phenomena.

US2011/0020929 describes a solution applied to a microfluidic system to generate flow from a well to an underlying compartment, activating an incoming flow in an upper compartment. The fluid movement is achieved by using a porous medium.

There is a need for devices which allow the dynamic culture of biological cells and tissues, allowing the dynamic release of external factors, nutrients or drugs, with the aim of recreating the conditions of the physiological microenvironment within the culture wells and thus providing a significant contribution in the study of drug resistance.

In particular, there is a need for devices which allow the controlled discharge of fluids from wells to be managed on a large scale and, if required, the controlled recovery thereof.

### DESCRIPTION

In an embodiment, a discharge system is claimed, which allows the regulation of the output flow rate of a fluid from a cell culture well.

In an embodiment, a well is claimed, comprising a discharge system and, optionally, an inlet, where said inlet, where present, and a portion of said discharge system comprise open tubes of capillary size, i.e., having a hydraulic diameter between about 0.05 and about 2 mm, and respectively allow the fluid to enter and exit in/from said well.

In an embodiment, a multiwell plate comprising the discharge system according to the present invention is claimed. Said multiwell plate finds convenient application in microfluidic platforms.

The present invention also relates to a method for perfusing and optionally collecting an eluate from a cell culture well.

### DESCRIPTION OF THE DRAWINGS

FIG. 1: (A) an embodiment of the discharge system according to the present invention. (B) a further embodiment of the discharge system according to the present invention. (C) perspective view of the frustoconical distal portion in the discharge system according to the embodiment of panel B.

FIG. 2: an embodiment of wells in a multiwell plate comprising an inlet and an outlet according to the present invention. (A) multiwell plate; (B) perspective view, (C) front view and (D) top view of a single well.

FIG. 3: Diagram of the method according to the present invention: (A) filling the well; (B) reaching the maximum fill level and starting formation of the discharge drop; (C) releasing the discharge drop. In the upper panel, the well comprises a “micro-droplet” containing organoids. In the lower panel, the well comprises adhering culture cells.

FIG. 4: detail of a movable element of a “micro-droplet” plate in three successive operating steps. (A) step 1, loading face up (top) and, alternatively, face down (bottom) with micro-droplet formation; (B) step 2, micro-droplet isolation; (C) step 3, micro-droplet positioning in the well and culture/treatment.

FIG. 5: perspective view of a micro-droplet plate in culture/treatment position above a multiwell plate.

FIG. 6: Average volumes of the single drop generated with discharge system comprising an outlet having a frustoconical distal portion with outlet opening having a diameter of 1.5, 2, 2.5 mm, respectively, with (A) distilled water; (B) PBS (Phosphate Buffer Solution); (C) culture medium.

FIG. 7: Release times of the single drop with discharge system comprising an outlet, as a function of the input fluid flows [Q] through inlets in the single well, with reference to three different diameters of the outlet opening of the frustoconical distal portion, 1.5 mm (upper quadrant), 2 mm (central quadrant), 2.5 mm (lower quadrant), and with different fluids: (A) distilled water, (B) PBS (Phosphate Buffer Solution), (C) culture medium.

FIG. 8: embodiment of a discharge system comprising an outlet and a collection system, (A) front view; (B) perspective view.

### DETAILED DESCRIPTION OF THE INVENTION

The present invention relates to a discharge system conveniently used in cell culture wells.

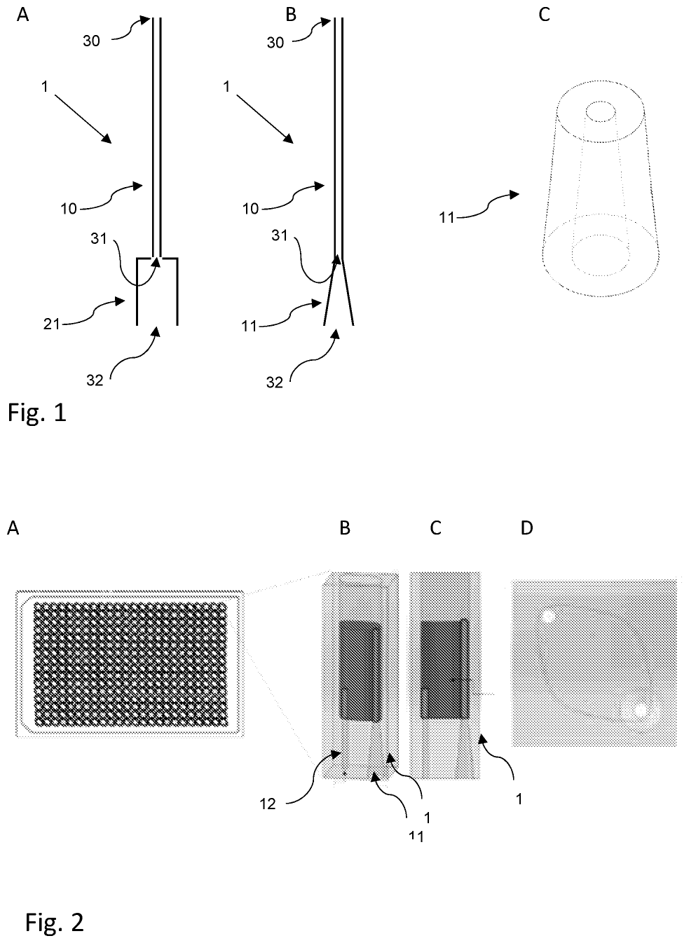

In an embodiment, said discharge system comprises an outlet 1 comprising, with reference to FIG. 1, a proximal portion 10 and a distal portion 11, 21. Said proximal portion 10 comprises a capillary tube which engages on said distal portion 11, 21. Said capillary has an inlet 30 and an outlet 31, where said outlet 31 engages on said distal portion 11, 21. Said distal portion on which said capillary is engaged is open and has an outlet opening 32 with a greater hydraulic diameter than the hydraulic diameter of said capillary.

In an embodiment, the hydraulic diameter of said capillary is between 0.05 mm and 2 mm, or between 0.10 mm and 1.3 mm, or between 0.20 mm and 1 mm, and the hydraulic diameter of said outlet opening is between 1 and 4 mm, or between 1.1 and 3 mm, or between 1.3 and 3 mm.

In an embodiment, with reference to FIG. 1A, said distal portion is cylindrical.

In an embodiment, with reference to FIG. 1B, said distal portion is tapered upwards, i.e., said distal portion has an inlet opening at the engagement of said capillary and an outlet opening which is distal with respect to said engagement, and said outlet opening 32 has a greater hydraulic diameter than the hydraulic diameter of said inlet opening, where the hydraulic diameter of said inlet opening is the hydraulic diameter of said capillary.

In an embodiment, said distal portion 11 is the frustoconical structure depicted in FIG. 1C. Said frustoconical structure is characterized by having an upper base, which is said inlet opening, having a diameter between 0.05 mm and 2 mm, or between 0.10 mm and 1.3 mm, or between 0.20 mm and 1 mm and a lower base, which is said outlet opening, having a diameter between 1 and 4 mm, or between 1.1 and 3 mm, or between 1.3 and 3 mm, where said diameter of said outlet opening is always greater than the diameter of said inlet opening.

Said capillary is an empty tube, free for the passage of a fluid flow and possibly of a particulate as well, as long as the dimensions of said particulate are smaller than those of the tube itself having a maximum hydraulic diameter of about 2 mm.

Advantageously, a capillary which is an empty tube is sterilizable and reusable.

The solution suggested here generates a flow which is globally from the top downwards. In fact, the capillarity only intervenes during the filling step, then it is the action of gravity which moves the fluid. The difference in height between the free surface and the meniscus of the drop generates a pressure which overcomes the resistance exerted by the meniscus of the drop, precisely because the wider diameter of said outlet opening than the diameter of said inlet opening makes said pressure higher than said resistance.

The present invention further relates to a well comprising a discharge system and optionally an inlet, where said discharge system comprises the outlet described above, or it is the discharge system described above.

With reference to FIG. 2, said well, in an embodiment, is a well of a multiwell plate, for example of a multiwell plate with 96 wells, or 384 wells, or 1536 wells.

At least one discharge system is conveniently housed in said well, comprising an outlet 1 according to the present invention, where the inlet of the capillary of said outlet is inside said well. In an embodiment, said capillary is at least partially positioned inside the well. In an embodiment, said capillary is at least partially integrated in the wall of said well.

Said inlet of said capillary is positioned inside said well at a convenient height. Said convenient height is the maximum height reachable by the fluid volume contained in said well.

Optionally, a portion of said distal portion 11, 21 of said outlet 1 is outside said well, below the base thereof.

In an embodiment, said well also comprises an inlet 12. Said inlet 12 is a capillary tube having an inlet port which is outside said well and an outlet port inside the well itself. In a preferred form, said inlet capillary is positioned such that said inlet port is outside, preferably below said well and said outlet port is inside said well. In an embodiment, said inlet is, at least in part, inside said well; in a further embodiment said capillary is at least in part integrated in the wall of said well.

Said well comprising said outlet and, optionally, said inlet, has any suitable shape. For example, it has a circular, or square, or elliptical, or rhomboidal section. Conveniently, where said well is one of the wells of a multiwell, said shape is such that the distance between the center of adjoining wells is equal to the distance which is measured between the center of adjoining wells in standard multiwell plates, so that the device is compatible with standard instrumentation.

In an embodiment, with reference to FIG. 2D, said well has a rhomboidal section, preferably with rounded corners.

In this embodiment, said inlet and said discharge system are conveniently positioned at the diametrically opposite vertices of said rhombus, along the largest diameter. This arrangement favors a flow dynamic which is particularly advantageous for the culture dynamics. Moreover, while keeping the standard infra-well distance, the rhomboidal shape allows making the best use of the available volumes, leaving volumes outside the well itself in which to house the microfluidic channels required by the device.

In an embodiment, where said well is made of hydrophilic materials, the inner walls of said well comprise grooves which allow controlling the behavior of the fluid meniscus.

Advantageously, the system suggested here is capable of generating an emptying flow, using passive components of a size compatible with microplates. Said flow is interrupted when the height of the fluid in the well drops with respect to the level of the outlet channel mouth, and this allows interrupting the perfusion of the well, keeping it full, and then resuming it at a later time without disturbing the system.

Where the size of the drop does not depend solely on the geometry of the distal portion, but also on the interaction between fluid and material with which the system (well, outlet channel and/or distal portion) is made, whether hydrophobic or hydrophilic, said drop may become too large. For example, it can come to have a volume almost equal to the volume of the well from which it originates. In this case, the detachment thereof is cause of instability, and the variability of the system increases, in addition to being scarcely predictable. For example, an overly large drop may adhere to the side walls of the collection well and thus not reach the bottom thereof, as would be desired for a proper controlled collection of the eluate.

In this regard, the present invention further relates to a discharge system which also comprises a collection system. Said collection system allows generating, in a repeatable and controlled manner, the detachment of the drop upon reaching the desired volume, where said volume is defined by the distance D between said outlet opening of said outlet and the same collection system.

Upon reaching the desired volume, the drop comes into contact with the collection system, which is a hollow capillary tube or, optionally, filled with a porous medium which induces the instantaneous detachment thereof and leads it to the bottom of a collection well, where available.

Said collection system is initially filled by gravity and capillarity. Once filled, the flow is induced by the subsequent drops by gravity. The hydraulic diameter of said collection system is of comparable dimensions with those of the outlet opening of the outlet.

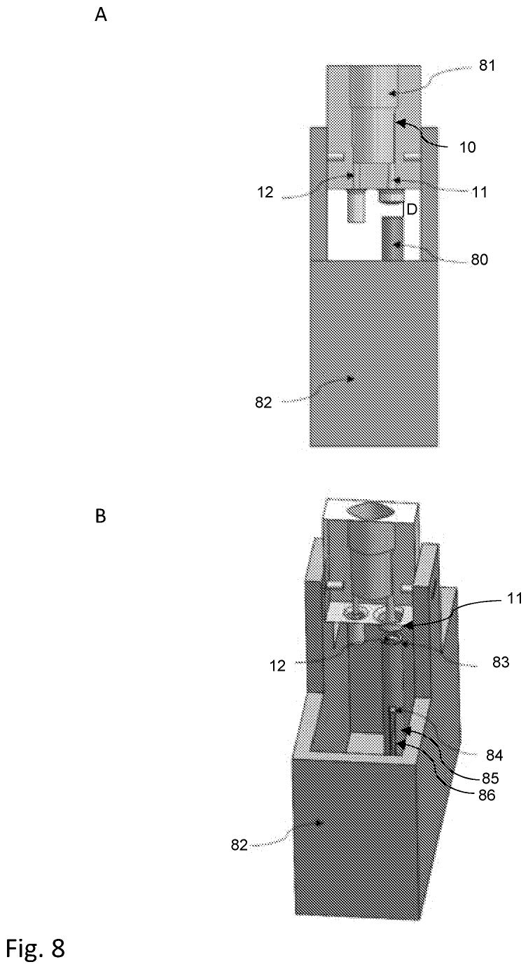

Referring to FIGS. 8A, 8B in said well 81 said fluid enters through said inlet 12 and exits through said outlet comprising a proximal portion 10 and a distal portion which is a frustoconical distal structure 11. Said collection system 80 is conveniently positioned at a distance D from the outlet opening of the outlet. Said collection system is conveniently positioned below said outlet, preferably in a direction parallel to the direction of the capillary tube forming the proximal portion 10 of said outlet.

Said collection system comprises an upper end, referred to as a perforated cup 83, and a lower end, referred to as an outlet hole 84. Said perforated cup interfaces with the drop generated by said outlet and has dimensions equal to or greater than the drop itself.

In an embodiment, the system also comprises a collection well 82.

The eluate reaches the external environment or, where present, the collection well 82, through said outlet hole 84.

In an embodiment, said outlet hole 84 is positioned on the side wall of the collection system 80, which is at least partially in the collection well 82, where present. The outlet hole 84 is placed in a position such as to avoid being invaded by the fluid contained in the collection well itself.

In a preferred form, with reference to FIG. 8B, said collection system is supported by a support, for example a cylindrical support 85, which rests on the bottom of said collection well. Said cylindrical support conveniently comprises at least one groove 86, which facilitates the flow of the fluid towards said collection well, preventing it from adhering to the walls of said support.

The present invention further relates to a method for dynamically culturing cells and/or tissues.

Said method comprises:
Providing at least one well, where said well comprises a discharge system and optionally an inlet, where said discharge system comprises an outlet comprising a proximal portion and a distal portion, said proximal portion comprising a capillary which engages on said distal portion which is open and has an outlet opening with a greater hydraulic diameter than the hydraulic diameter of said capillary;
Providing at least one cell and/or tissue culture;
Seeding said culture in said at least one well;
Perfusing, through said inlet where present, with the desired input flow rates, fresh culture medium and/or actives and/or markers and/or washing buffers in said at least one well;
Keeping the dynamic culture for the desired timing;
Optionally, collecting the discharge liquid exiting from said discharge system for subsequent analysis.

The method according to the present invention preferably uses input flow rates between 0 and 100 μl/min, or between 0.1 and 60 μl/min, or between 0.5 and 50 μl/min, or between 1 and 25 μl/min.

In the embodiment where the discharge system comprises an outlet and the collection system, upon reaching the desired volume of the drop exiting from said outlet, defined by the distance D between said outlet opening of the outlet and the perforated cup of said collection system, the drop comes into contact with the collection system which induces the instantaneous detachment thereof and leads it, by means of the outlet hole, in the external environment, or in the collection well.

The flow can be controlled by varying the distance D. Where the distance D is less than the diameter of the outlet opening of the outlet, an exiting continuous flow is obtained. Said input flow rates may vary while performing the method. Merely by way of example, said method can include a step in which said input flow rate is 0, and the culture is incubated in the fluid volume present in the well, a further step in which said input flow rate is about 10 μl/min, in which said fluid volume contained in the well is rapidly replaced, a further step in which said input flow rate is about 5 μl/min, in which said fluid volume contained in the well is slowly replaced. Said steps can be repeated cyclically.

In an embodiment, said outlets are previously loaded with fluid, for example by exerting a vacuum or a pressure below or above them, respectively. In this embodiment, after said outlet is full and the first drop is generated, the system operates passively.

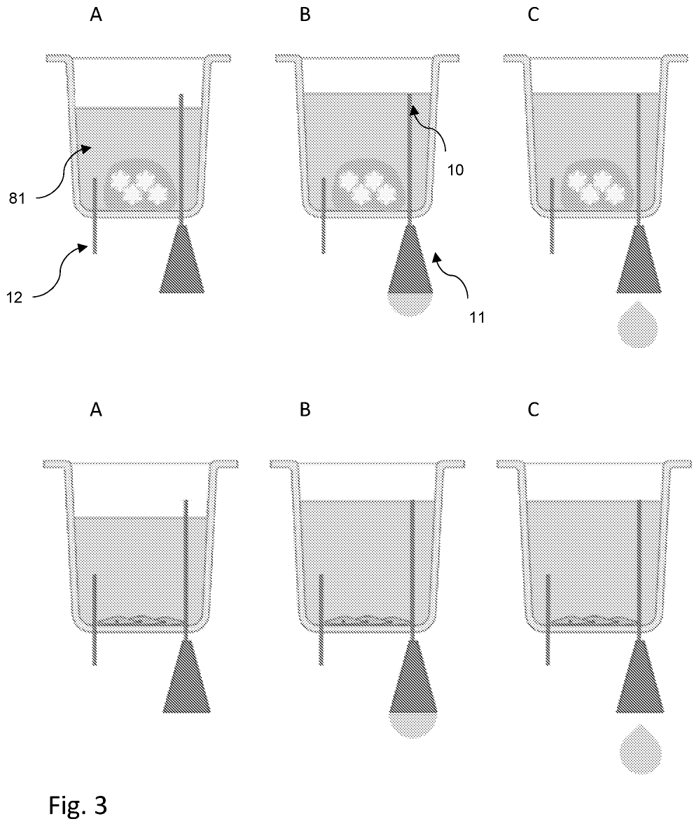

With reference to FIG. 3, in a first step, A, the fluid is introduced through said inlet 12 into the well 81. In a subsequent step, where said fluid reaches in said well the height in which the inlet of said capillary 10 of said outlet is located, B, said fluid enters said outlet and, by virtue of the presence of the frustoconical distal structure 11, by capillarity and gravity is dragged towards the outlet. Upon the formation of a drop, C, when said force exceeds the critical mass, a fraction of said fluid which was contained in said well is released from said discharge system and is possibly available for collection. That is, when the weight force exerted on the drop exceeds the surface tension, the drop detaches from said distal portion 11.

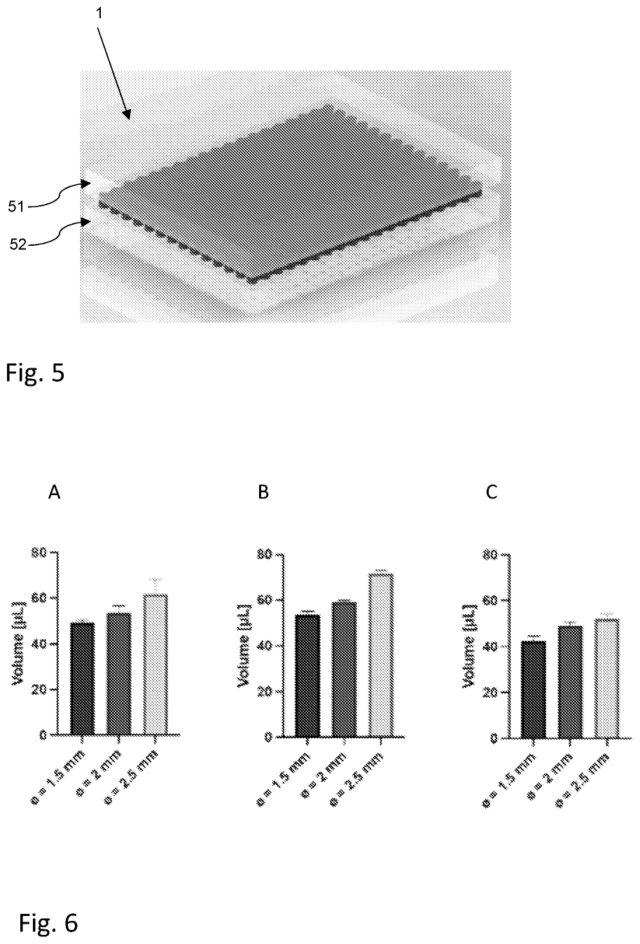

In an embodiment, the system according to the present invention is used in a microfluidic platform, where said platform 1, with reference to FIG. 5, comprises two layers: an upper layer 51 and a lower layer 52.

In an embodiment, said microfluidic platform is the microfluidic platform described in WO2021220173.

In an embodiment, said upper layer 51 is functional to the generation of matrix “micro-droplets” containing organoids, for example said upper layer is a “micro-droplet” plate as described in WO2021220173, where said drops are suspended in the wells included in the lower layer 52 which is for example a multiwell plate.

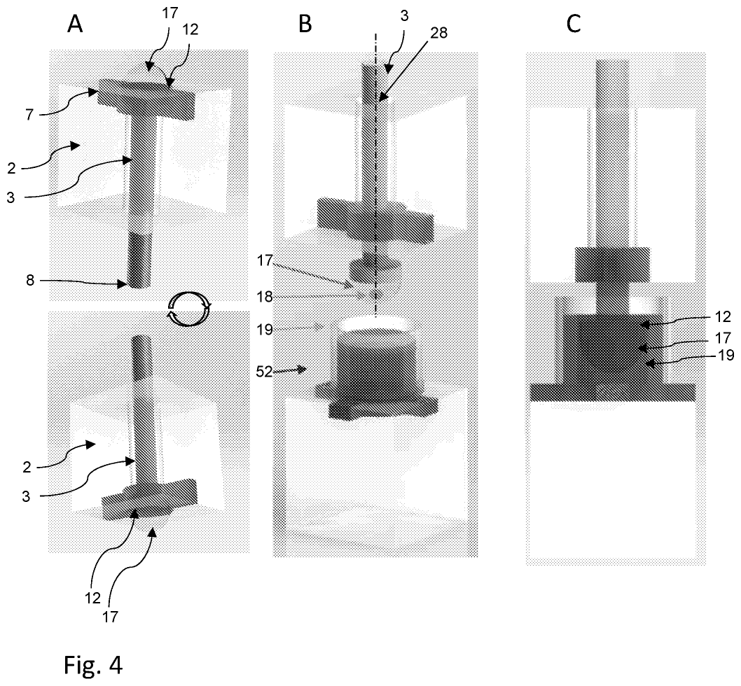

By way of example, in this embodiment the method according to the present invention comprises, with reference to the operating steps outlined in FIG. 4:
Step 1, loading with micro-droplet formation (FIG. 4A)

A “micro-droplet” plate, comprising movable elements 3 in resting position, is positioned face up (FIG. 4A, upper panel) and the integrated microfluidic circuit is loaded with a fluid, where said fluid is a SOL. After loading, said fluid is free to flow and goes to occupy the hydrophilic areas present in the integrated microfluidic circuit, i.e., the microchannels and the hydrophilic surfaces with elements in relief 12. Said fluid, by virtue of said hydrophilic surfaces with elements in relief 12, expands, forming SOL caps 17 protruding from the “micro-droplet” plate. When said integrated microfluidic circuit is conveniently filled by said fluid, said fluid is gelled, coming to obtain a “micro-droplet” plate where the hydrophilic areas of the integrated microfluidic circuit are homogeneously occupied by a gel. In particular, the fluid which is located at each of the hydrophilic surfaces with elements in relief 12 which has formed said SOL caps 17 gels, forming a “micro-droplet” of gel 17.

In an embodiment, said micro-droplet plate is turned face down (FIG. 4A, lower panel), for the gelling process, where it is observed that the face-down gelling advantageously leads to obtaining micro-droplets of gel of greater volume than the micro-droplets of gel obtained by gelling with the face-up micro-droplet plate.
Step 2, micro-droplet isolation (FIG. 4B)

The micro-droplet plate with the hydrophilic areas of the integrated microfluidic circuit homogeneously occupied by fluid in gel phase is face down and is conveniently positioned above a plate for cell cultures 52 containing a culture medium 19. The movable elements 3, loaded with said gel drops 17, also independently of one another, reach the operating position, or emerge from said fixed element 2 towards the upper face thereof.

Advantageously, said plate for cell cultures 20 is a multiwell plate, where at least one well of said plate for cell cultures is conveniently located below a movable element 3 loaded with said gel drop 17.
Step 3, immersion (FIG. 4C)

The gel micro-droplet 17 on said movable element 3 is immersed in the culture medium 19. In an embodiment, said gel micro-drople” 17 remains hanging on the movable element 3 and immersed in said culture medium for the entire duration of the treatment.

In another embodiment, the movable element 3, retracting from said operating position to said resting position, releases said gel “micro-droplet” 17 in said culture medium 19.

Said “micro-droplet” 17, which contains cellular material, for example organoid, is thus kept in said culture medium for the entire duration of the treatment.
Step 4, treatment

Said well comprising the culture medium 19 comprises an inlet and an outlet according to the present invention. Through said inlet, fresh culture medium and/or actives and/or markers and/or washing buffers will be introduced for the entire duration of the treatment, with the desired times. Said perfusion is possible without disturbing the equilibrium of the culture, where the discharge of the fluid is regulated by said outlet.

The liquid discharged through said outlet is optionally collected on desired media for subsequent uses. Advantageously, said liquid, collected drop by drop, allows the separate collection thereof in distinct steps of said treatment.

The formation of drops for a system with capillary inlets and outlets, in wells of standard height, is ensured by the presence of the distal portion, the hydraulic outlet opening diameter of which is greater than the hydraulic diameter of the capillary. Said hydraulic outlet opening diameter is conveniently selected based on the surface tension of the fluid used, the manufacturing/coating material of the discharge system itself, the input flow rate.

The adjustment of the discharge system is a passive adjustment where, once the perfusion is started, it proceeds autonomously and passively.

Advantageously, the system described herein allows a high reproducibility of the method, versatility and uniformity of the volume of the output liquid.

The system described herein is modular and as such compatible with devices typically used in cell biology laboratories.

Advantageously, the system allows collecting the eluates over time from each individual well and keeping them mutually separate.

The following examples have the sole purpose of exemplifying the solution, are not to be intended as limiting it in any manner, the scope of which is determined by the following claims.

### EXAMPLES

### Example 1: Calculating the Volume and Release Time of Exiting Drops

The experiment was conducted in a well according to the present invention comprising an inlet and an outlet. Said outlets comprise a frustoconical distal portion and, under different experimental conditions, three distal portions were tested which differ from one another by the outlet opening diameter. Outlets with distal portions with said outlet opening diameter not exceeding 1.5 mm, 2 mm or 2.5 mm were tested, with the same inlet opening diameter being 0.13 mm.

Said wells are loaded with a controlled input flow rate of 10 μl/min. In a stationary step, the fluid level inside the well remains stable. Excess fluid in input from the inlet is drained from the outlet and collected in an underlying eppendorf. Every 10 drops collected, the eppendorfs are weighed so that the average weight of the individual drops can be estimated as equal to one tenth of the measured weight. Knowing the density of the fluid, under the same temperature conditions, the volume of the individual drops is then calculated. The experiment was repeated three times for each configuration.

The experiment was repeated using three different fluids: distilled water, PBS, and culture medium. The results obtained are reported in FIGS. 6A, B and C, respectively, and show how said discharge system works with all the fluids tested and the volumes vary as a function of the fluid, as they have different surface tension. The drop volumes further increase as the outlet opening diameter of the distal portion of said outlet increases.

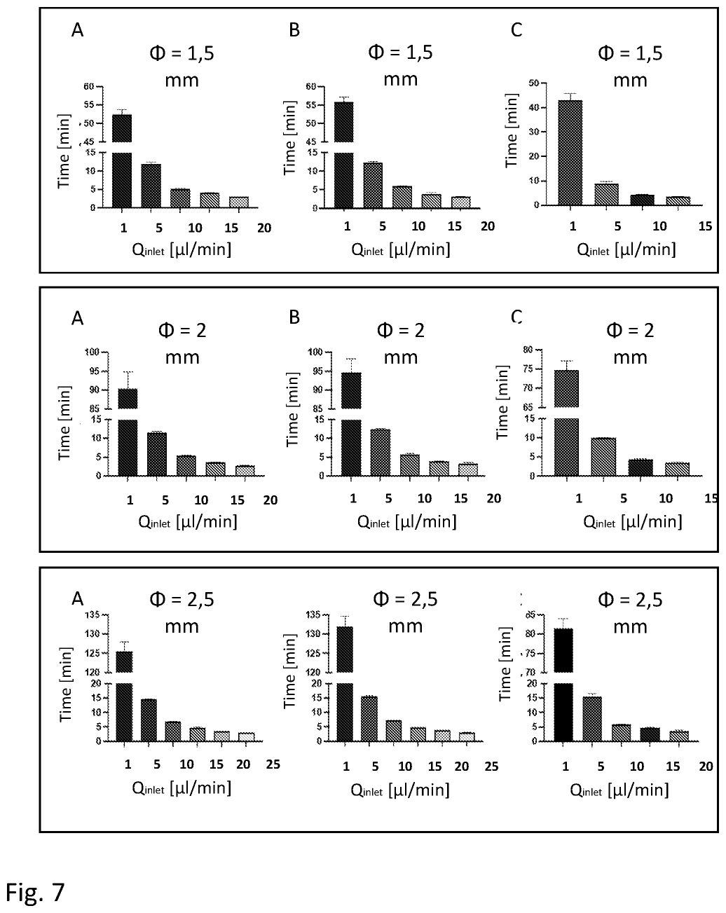

In a similar experiment, the release times of the individual drops under different experimental conditions were measured. The results reported in FIG. 7 show how, with the same fluid flow entering in the well, the release times of the individual drops, i.e., the time elapsing between the release of one drop and the next one, increase as the outlet opening diameter of the distal portion increases and decrease as the surface tension decreases.

By increasing the fluid flow entering in the well, the release times of the individual droplets in the three experimental models decrease until a flow rate threshold is reached, which is greater for the experimental model having the largest outlet opening diameter. Above said flow rate threshold, the discharge system is no longer capable of compensating for the incoming flow, and the level of fluids in the well rises, as shown by the graphs in FIG. 7.

1. A discharge system for cell culture wells comprising an outlet comprising a proximal portion and a distal portion, wherein said proximal portion comprises a capillary which engages on said distal portion, said capillary having an inlet and an outlet, wherein said outlet engages on said distal portion which is open and has an outlet opening with a greater hydraulic diameter than the hydraulic diameter of said capillary.

2. The discharge system according to claim 1, wherein the hydraulic diameter of said capillary, which is the inlet opening, is between 0.05 mm and 2 mm, or between 0.10 mm and 1.3 mm, or between 0.20 mm and 1 mm, and the hydraulic diameter of said outlet opening is between 1 and 4 mm, or between 1.1 and 3 mm, or between 1.3 and 3 mm.

3. The discharge system according to claim 1, wherein said distal portion is tapered from the bottom upwards.

4. The discharge system according to claim 3, wherein said distal portion is a frustoconical structure having an upper base which is the inlet opening having a diameter between 0.05 mm and 2 mm, or between 0.10 mm and 1.3 mm, or between 0.20 and 1 mm and a lower base which is the outlet opening having a diameter between 1 and 4 mm, or between 1.1 and 3 mm, or between 1.3 and 3 mm.

5. The discharge system according to claim 1, also comprising a collection system, wherein said collection system is a tube comprising an upper end, referred to as a perforated cup, and a lower end, referred to as an outlet hole, characterized in that said perforated cup is positioned at a distance D from said outlet opening of said outlet, wherein said distance D defines the volume of the drop. Filed Herewith

6. The discharge system according to claim 1, wherein said wells are wells of multiwell plates.

7. A cell culture well comprising a discharge system according to claim 1.

8. The well according to claim 7, also comprising an inlet which is a capillary having an inlet port housed outside the volume defined by said well and an outlet port housed inside the volume defined by said well.

9. The well according to claim 1, having a rhomboidal section, said well comprising inlets and outlets positioned at the diametrically opposite vertices of said rhombus, along the larger diameter thereof.

10. The well according to claim 8, wherein said outlets and optionally inlets are positioned inside the volume defined by said well or are integrated in the walls thereof.

11. The well according to claim 7, further comprising
a collection system, wherein said collection system is a tube comprising an upper end, referred to as a perforated cup, and a lower end, referred to as an outlet hole, characterized in that said perforated cup is positioned at a distance D from said outlet opening of said outlet, wherein said distance D defines the volume of the drop.

12. The well according to claim 7, also comprising a collection well for the fluid exiting from said collection system.

13. A multiwell plate comprising a plurality of wells according to claim 7.

14. A method for dynamically culturing cells and/or tissues, wherein said method comprises:
Providing at least one well, wherein said well comprises a discharge system comprising an outlet, wherein said outlet comprises a proximal portion and a distal portion, said proximal portion comprising a capillary which engages on said distal portion which is open and has an outlet opening with a greater hydraulic diameter than the hydraulic diameter of said capillary;
Providing at least one cell and/or tissue culture;
Seeding said culture in said at least one well;
Perfusing, with the desired input flow rates, fresh culture medium and/or actives and/or markers and/or washing buffers in said at least one well;
Keeping the dynamic culture for the desired timing;
Collecting the discharged liquid exiting from said outlet for subsequent analyses.

15. The method according to claim 14, wherein said well also comprises an inlet and said perfusion occurs through said inlet, with input flow rates between 0 and 100 μl/min, or between 1 and 60 μl/min, or between 0.1 and 60 μl/min, or between 0.15 and 50 μl/min, or between 1 and 25 μl/min.

16. The method according to claim 14, wherein said discharge system also comprises a collection system.
